(19) United States
(12) Patent Application Publication
(10) Pub. No.: US 2024/0011014 A1
(43) Pub. Date: Jan. 11, 2024

(54) IN-TIP MAGNETIC-BASED MAGNETIC BEAD PROCESSING

(71) Applicants: Illumina, Inc., San Diego, CA (US); Illumina Cambridge Limited, Cambridge (GB)

(72) Inventors: Gianluca Artioli, Cambridge (GB); Brian Mather, San Diego, CA (US); Dave Jones, Huntingdon (GB)

(73) Assignees: Illumina, Inc., San Diego, CA (US); Illumina Cambridge Limited, Cambridge (GB)

(21) Appl. No.: 18/216,859

(22) Filed: Jun. 30, 2023

Related U.S. Application Data
(60) Provisional application No. 63/357,210, filed on Jun. 30, 2022.

(51) Int. Cl. C12N 15/10

(52) U.S. Cl. CPC C12N 15/1013

(57) ABSTRACT

A method may be implemented for in-tip flow-through magnetic bead processing. A biological solution may include a plurality of magnetic beads suspended therein. The biological solution may be introduced to a tube via an opening in a tip portion of the tube. The tip portion of the tube may include a magnetizable material arranged in a flow path of the biological solution. The magnetizable material may include a ferromagnetic matrix or a wire within the tip portion. A magnetic field may be applied proximate to the tip portion of the tube using an electromagnetic coil. The electromagnetic coil may be wound around the tip portion. The biological solution may be removed from the tube, for example, via the opening in the tip portion. The plurality of magnetic beads may be captured within the magnetizable material in the tip portion using the magnetic field.

### CROSS-REFERENCE TO RELATED APPLICATIONS

This application claims priority to U.S. provisional patent application no. 63/357,210, filed Jun. 30, 2022, which is incorporated herein by reference in its entirety.

### BACKGROUND

During library preparation for deoxyribonucleic acid (DNA) sequencing, several steps require the use of magnetic beads. For example, magnetic beads are often used to perform size selection, clean up, and enrichment. Standard magnetic pull-down stations are well known and are used to separate and hold beads during manual manipulations. In standard magnetic pull-down stations, a strong permanent magnet is raised or lowered against the bottom of a microplate.

Standard magnetic pull-down stations present some challenges. First, the automated tip head in a standard magnetic pull-down station may require precise alignment to reach the bottom of a 96-well plate without disturbing a magnetic pellet on a side. A standard magnetic pull-down station may also require a high dead volume (e.g., approximately 20%) with standard automation alignment.

### SUMMARY

Systems, methods, and apparatus are described herein for in-tip flow-through magnetic bead processing. A method of in-tip flow-through magnetic bead processing may include providing a biological solution. The biological solution may include a plurality of magnetic beads suspended therein. The biological solution may be introduced to a tube via an opening in a tip portion of the tube. The tip portion of the tube may include a magnetizable material arranged in a flow path of the biological solution. The magnetizable material may include a ferromagnetic matrix or a wire (e.g., coiled, intercrossed, etc.) within the tip portion. The ferromagnetic matrix may include a steel wool, a nickel wool, a cobalt wool, an iron wool, and/or another ferromagnetic material wool. The steel wool may have a mass of 100 mg or less (e.g., 10 mg or less). A magnetic field may be applied proximate to the tip portion of the tube using an electromagnetic coil. The electromagnetic coil may be wound around the tip portion. The biological solution may be removed from the tube, for example, via the opening in the tip portion. The plurality of magnetic beads may be captured within the magnetizable material in the tip portion using the magnetic field.

The magnetic field may be applied proximate to the tip portion by introducing a flow of current through the electromagnetic coil. The magnetic field proximate to the tip portion may be removed by interrupting the flow of current through the electromagnetic coil. A second biological solution may be introduced to the tube via the opening in the tip portion such that the captured plurality of magnetic beads are suspended in the second biological solution.

Another method of in-tip flow-through magnetic bead processing may include providing a biological solution. The biological solution may include a plurality of magnetic beads suspended therein. The biological solution may be introduced to a tube via an opening in a tip portion of the tube. The tip portion of the tube may include a magnetizable material layer applied to an inner surface of the tip portion. The magnetizable material layer may include a ferromagnetic stainless steel. The ferromagnetic stainless steel may be a 300 series austenitic stainless steel (e.g., 303 stainless steel, 304/304L stainless steel, 316/316L stainless steel, 321 stainless steel, or 347 stainless steel), a 400 series martensitic stainless steel (e.g., 410 stainless steel, 416 stainless steel, or 440C stainless steel), or a duplex stainless steel (e.g., 2205/2207 stainless steel). For example, the ferromagnetic stainless steel may be a 416 grade stainless steel. A magnetic field may be applied proximate to the tip portion of the tube, for example, by placing a magnet proximate to the tip portion. The biological solution may be removed from the tube via the opening in the tip portion. The plurality of magnetic beads may be captured against the magnetizable material layer in the tip portion using the magnetic field as the biological solution is removed from the tube.

The magnetic field proximate to the tip portion may be removed by moving the magnet away from the tip portion. A second solution (e.g., buffer solution, biological solution, and/or the like) may be introduced to the tube via the opening in the tip portion such that the captured plurality of magnetic beads are suspended in the second solution. The tip portion may be made from the ferromagnetic material. The magnetic field is applied proximate to the tip portion by applying current through an electromagnetic coil that is wound around the tip portion. The magnetic field proximate to the tip portion may be removed by interrupting the current through the electromagnetic coil.

### BRIEF DESCRIPTION OF DRAWINGS

FIG. 1 is an illustration depicting an example magnetic bead pull down station.

FIG. 2 is an illustration depicting an example in-tip magnetic based magnetic bead process.

FIGS. 3A-3D depict cross-section views of example in-tip flow-through magnetic bead processing systems.

FIG. 4 is a flowchart of an example process of capturing magnetic beads using an in-tip flow-through magnetic bead processing system.

FIG. 5 illustrates a block diagram of an example computing device that can be used with one or more of the example in-tip magnetic-based magnetic bead processing systems shown in FIGS. 3A-3D and/or to perform the example process shown in FIG. 4.

### DETAILED DESCRIPTION

Magnetic beads have a number of applications in library preparation for deoxyribonucleic acid (DNA) sequencing. For example, magnetic beads may be used for DNA isolation and purification, DNA size selection, RNA isolation and purification, Tagmentation, enrichment, and normalization. The magnetic beads may be introduced in a biological solution. The biological solution may comprise one or more biological mediums (e.g., such as blood, plasma, saliva, etc.) and/or one or more buffers. For example, the magnetic beads may be added to a biological solution in wells of a sample plate (e.g., well plate) at a predetermined bead-to-DNA ratio. The sample plate may comprise a microtiter plate with multiple wells. Each of the wells are used as individual reaction vessels (e.g., such as test tubes) during multiple steps in library preparation. A microtiter plate may be a flat plate with between 6 and 9600 individual wells (e.g., between 6 and 1536 individual wells) that are arranged in a 2×3 rectangular matrix. A picotiter plate may be a smaller version of the microtiter plate. The picotiter plate may enable parallel sequencing of 1.7 million separate DNA fragments.

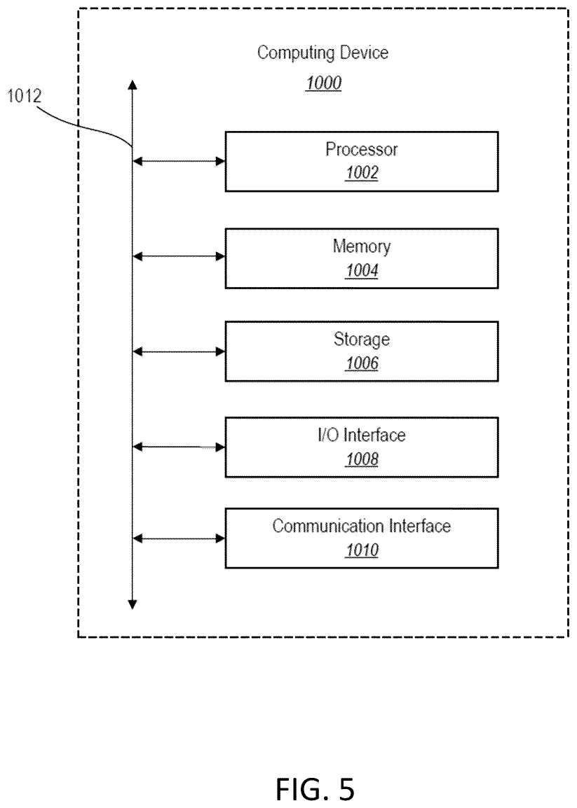

In examples, magnetic beads (e.g., paramagnetic beads) may be used to separate items (e.g., such as target molecules) from a solution. When target molecules are attached to the magnetic beads, complexes of target molecules and nucleic acid ligands may be partitioned from the solution (e.g., mixture) by the application of a magnetic field proximate to the wells. As described herein, the magnetic field may be applied by one or more magnets located proximate/adjacent to the walls of each well and/or an electromagnetic coil surrounding each of the wells. When the magnetic fields are activated (e.g., by a computing device such as the computing device 1000 shown in FIG. 5), the magnetic beads may be held to the sides (e.g., inner surfaces) of the wells. The magnets and/or electromagnetic coil may be an integral part of the workstation (e.g., that comprises the wells) or attached to a cover that is lowered over the workstation (e.g., by a robotic manipulator). When the magnets and/or electromagnetic coil are attached to a cover, the cover may be configured such that the magnets and/or electromagnetic coil can be placed adjacent to the wells without blocking access to the wells themselves. In this way, the wells may be accessible by pipetting and/or aspirating units when the cover is in place. Following magnetic field activation, liquid can be aspirated from the wells, followed by the addition of wash solutions.

When the magnetic fields are deactivated and/or when the cover is removed, the magnetic beads may become resuspended in the solution. The cover may be stored on a work surface. In examples, the magnets in the cover may be permanent magnets and withdrawing the cover removes the influence of the magnets, and allows the magnetic beads to return into suspension.

In examples, a pipetting tool may dispense aliquots of the magnetic beads—with their bound target—to the individual wells of a microtitre plate located on the workstation. Each of the wells may already comprise an aliquot of a candidate mixture of nucleic acid ligands previously dispensed by the robotic manipulator. After dispensing the magnetic beads, the robot may mix the contents of each well (e.g., by removing and reintroducing the contents of the wells several times). The microtitre plate may then be incubated at a preselected temperature on the workstation, for example, in order to allow nucleic acid ligands in the candidate mixture to bind to the bead-bound target molecule. The microtitre plate may be agitated, for example to ensure that the magnetic beads remain in suspension.

After incubation for a suitable time, the cover (e.g., the magnetic separator cover) may be placed over the microtitre plate, for example, by the robotic manipulator. The magnetic beads may be held to the sides of the wells, and the aspirator tool may remove the solution comprising unbound candidate nucleic acids from the wells. A washing solution, such as a low salt solution, may then be dispensed into each well by the pipetting tool(s). The magnetic beads may be released from the side of the wells by withdrawing the magnetic separator cover or deactivating the electromagnets/electromagnetic coil, then resuspended in the wash solution by agitation and mixing. The magnetic separator cover may be placed over the plate again, and the wash solution may be aspirated. This wash loop can be repeated for a pre-selected number of cycles. At the end of the wash loop, the magnetic beads may be held to the sides of the empty wells, for example, by the magnetic field generated by the electromagnets, electromagnetic coil, and/or permanent magnets.

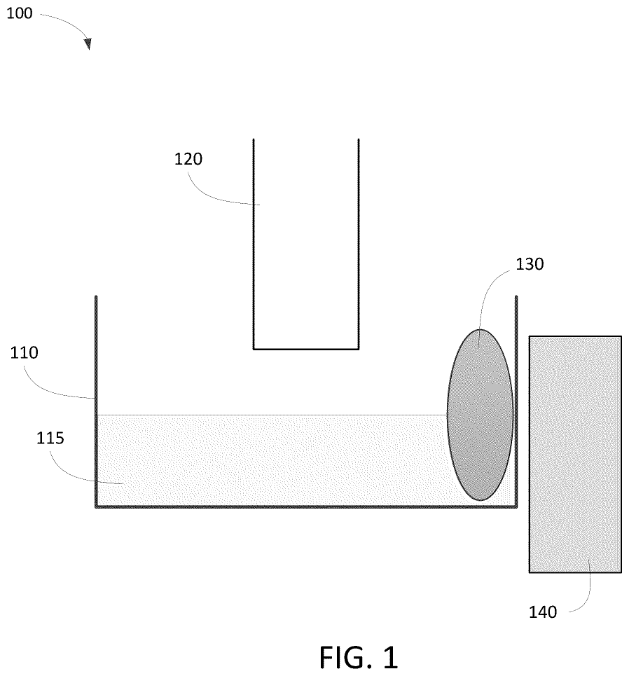

FIG. 1 illustrates a cross-section view of an example bead wash station 100. The bead wash station 100 may be a pull down bead wash station. The bead wash station 100 may comprise a reservoir 110, a tube 120, and a magnet 140. The relative location of the magnet 140 with respect to the reservoir 110 may be adjusted, for example, to change a magnetic field proximate to the reservoir 110. For example, the magnet 140 may be moved between a location proximate to (e.g., adjacent to) the reservoir 110 and a location distal from (e.g., away from) the reservoir 110.

The reservoir 110 may be configured to receive and/or retain a solution 115 used in one or more DNA sequencing procedures, for example, from the tube 120. The solution 115 may comprise a solid phase reversible immobilization (SPRI) solution, a buffer solution, a biological solution, and/or the like. One or more magnetic beads (e.g., such as magnetic bead 130) may be suspended in the solution 115 to perform the one or more DNA sequencing procedures. For example, the magnetic beads may be used in DNA purification, DNA extraction, DNA size selection, ribonucleic acid (RNA) Isolation, RNA Extraction, RNA Purification, Tagmentation, Enrichment, and/or Normalization.

When the solution 115 comprises one or more magnetic beads, the magnet 140 may be used to separate the magnetic beads from the solution 115. For example, when the magnet 140 is proximate to the reservoir 110, the magnetic bead 130 may be pulled towards a wall of the reservoir 110. The solution 115 may be removed from the reservoir 110 while the magnetic bead 130 is held in the reservoir 110, for example, by the magnetic force applied by the magnet 140. The solution 115 may be removed by the tube 120, another tube, and/or via a drain in the reservoir 110. In example pull-down bead wash stations, the reservoir 110 may be lowered away from the tube 120. In other example pull-down bead wash stations, the tube 120 may be lifted away from the reservoir 110.

The tube 120 (e.g., an automated tip head of the tube) may require precise alignment to reach the bottom of the reservoir 110 without disturbing the magnetic bead 130 on the wall of the reservoir 110. Eliminating the need for a high accuracy tube 120 (e.g., pipettor) may result in faster processing and considerable cost reduction. The reservoir 110 may require a large portion of dead volume (e.g., approximately 20%) to enable the precise alignment. The dead volume may define an excess liquid volume that remains in the reservoir 110, for example, to avoid disturbing the magnetic bead 130 on the wall of the reservoir 110.

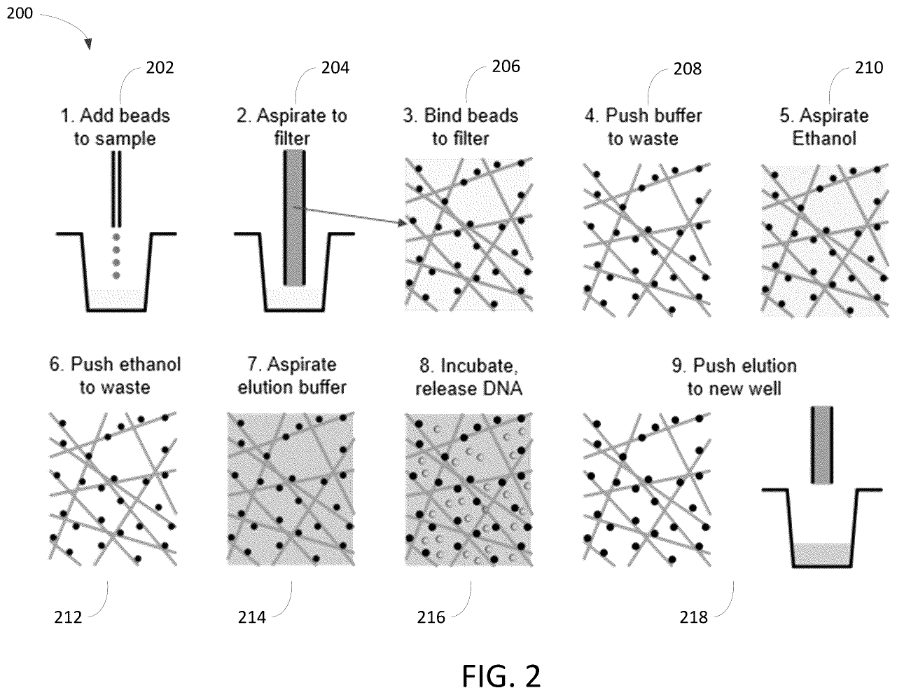

FIG. 2 depicts an example process 200 for using a flow-through magnetic bead processing system to remove magnetic beads from a solution. The example process 200 may be used for one or more DNA sequencing procedures, for example, such as DNA purification, DNA size selection, RNA Isolation, RNA Purification, Tagmentation, Enrichment, and/or Normalization. The example process 200 may be manual, automated, or a mix of manual steps and automated steps.

At 202, a plurality of magnetic beads may be introduced to a sample. The sample may comprise a solution that is held in a reservoir (e.g., such as the reservoir 110 shown in FIG. 1). The amount of magnetic beads added to the sample may be based on which DNA sequencing procedure is being performed. At 204, the solution may be aspirated through a filter. The filter may comprise a magnetizable material (e.g., such as a ferromagnetic matrix or a magnetizable surface) within a tube (e.g., such as the tube 120 shown in FIG. 1). When the solution is aspirated through the filter, the solution is pulled through the filter within the tube.

At 206, the magnetic beads within the solution may bind to the filter. For example, the filter media may be magnetized such that the magnetic beads are attracted to the filter media. At 208, the solution may be removed from the tube. When the solution is removed from the tube, the magnetic beads may remain bound to the filter.

At 210, ethanol may be aspirated to the filter. The magnetic beads may remain bound to the filter media as the ethanol is aspirated to the filter. For example, the magnet may remain proximate to the filter media/tube as the ethanol is introduced therein. The ethanol may be pulled through the filter media, for example, to cleanse the filter media and the magnetic beads. At 212, the ethanol may be removed from the tube. When the ethanol is removed from the tube, the magnetic beads may remain bound to the filter.

At 214, an elution buffer is aspirated to the filter. The magnetic beads may remain bound to the filter media as the elution buffer is aspirated to the filter. At 216, the elution buffer is incubated and DNA is released. At 218, the elution buffer may be removed from the tube.

Magnetic beads may be processed using a flow-through design. A flow-through magnetic bead processing system may overcome one or more challenges introduced by a pull-down magnetic bead processing system (e.g., such as the bead wash station 100 shown in FIG. 1). For example, the flow-through magnetic bead processing systems described herein may reduce total processing time and/or improve yields on library preparation automation.

In a flow-through magnetic bead processing system, a moving suspension of magnetic beads flows through a magnetic field, and beads are collected within a tube (e.g., a tip of the tube). One or more successive processing solutions may flow through the tube. Unless the fluid is moved at a very low flow rate, the drag forces of the fluid flow may dominate over the magnetic forces on the beads, resulting in low bead capture and retention. The magnetic field in a flow-through magnetic bead processing system must be strong enough to provide optimal bead capture, even at low flow rates. Trade-offs between processing speed, magnetic size, magnetic strength, and bead capture/retention may be performed. Solutions may be provided herein to reduce magnet size/strength requirements and accelerate processing speed while maintaining an optimal bead capture/retention.

The flow-through magnetic bead processing system may comprise a magnet-in-tip configuration, a magnetizable material in reagents configuration, a ferromagnetic matrix in tip configuration, and/or a ferromagnetic surface configuration. One or more of the flow-through magnetic bead processing systems may require application of a magnetic field. The magnetic field may be applied using an internal magnet, an external magnet, and/or an electromagnetic coil.

A ferromagnetic matrix magnetic bead system may be used for paramagnetic bead capture. For example, a ferromagnetic matrix, such as stainless-steel wool, may be incorporated inside a pipette tip. The ferromagnetic matrix may be within the flow path of a magnetic bead suspension. The ferromagnetic matrix magnetic bead system may comprise an external magnet that can magnetize the ferromagnetic matrix. When the ferromagnetic matrix is magnetized, paramagnetic beads may be captured on the surface of the ferromagnetic matrix. Ferromagnetic matrix magnetization may be temporary and may be reverted when the external magnet is removed (e.g., located distal from the pipette tip). The paramagnetic beads may be resuspended (e.g., fully resuspended) within a solution when the external magnet is removed. The reversibility of the magnetization and bead capture may be desirable for library preparation workflows where beads need to be captured and resuspended. Re-use of magnetic beads may decrease library preparation time and reduce laboratory storage requirements.

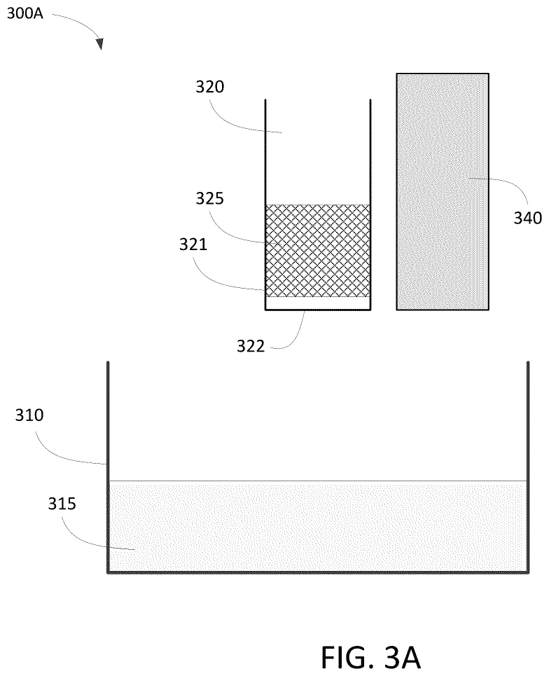

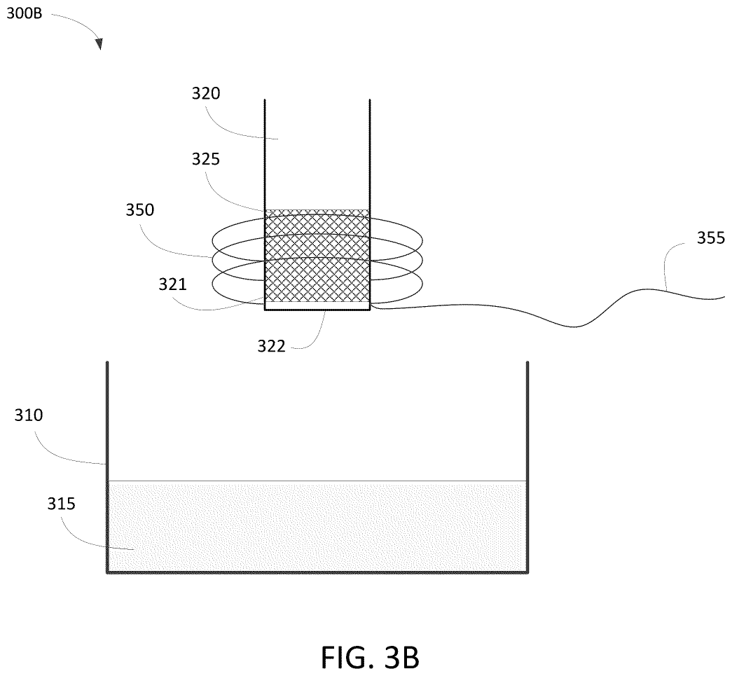

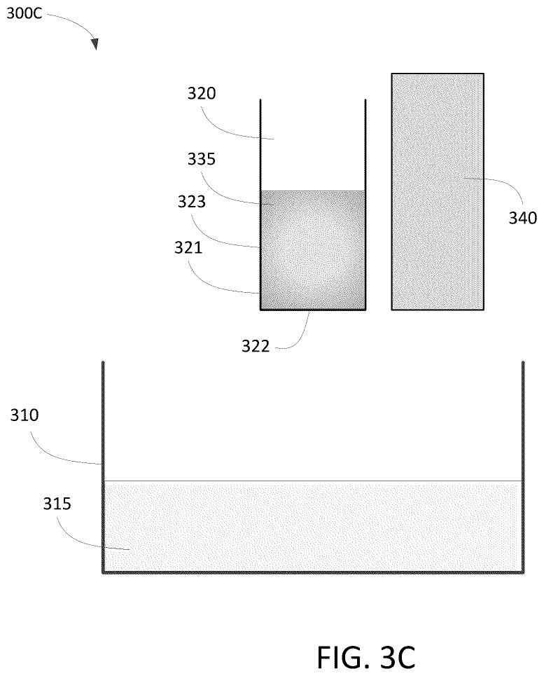

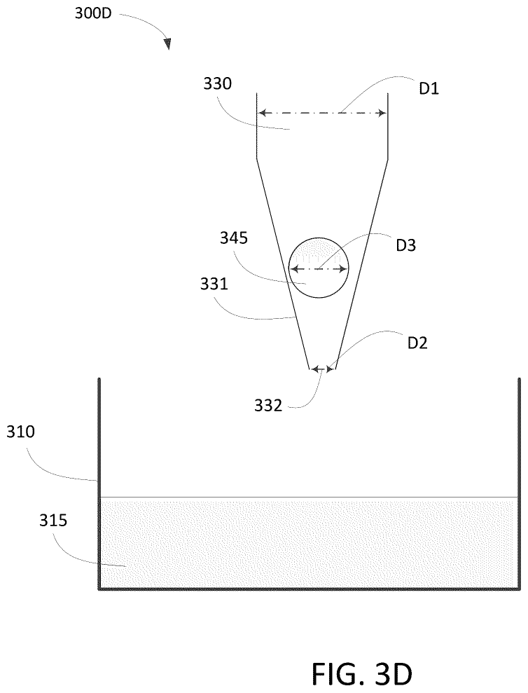

FIG. 3A illustrates a cross-section view of an example flow-through magnetic bead processing system 300A. The flow-through magnetic bead processing system 300A may comprise a reservoir 310 (e.g., a well in a plate), a tube 320, a ferromagnetic matrix 325, and a magnet 340. The tube 320 may comprise a 20 ul, 100 ul, 200 ul, or 1 mL pipette and/or a tube having a size between 0.02 mm and 5 mm. The reservoir 310 may be configured to receive and/or retain a solution 315 used in one or more DNA sequencing procedures, for example, from the tube 320. One or more magnetic beads (e.g., such as the magnetic bead 130 shown in FIG. 1) may be suspended in the solution 315 to perform the one or more DNA sequencing procedures. For example, the magnetic beads may be used in DNA purification, DNA size selection, RNA Isolation, RNA Purification, Tagmentation, Enrichment, and/or Normalization. The tube 320 may define a tip portion 321. The tube 320 may define an opening 322 in the tip portion 321. The opening 322 may be configured to receive the solution 315. For example, the solution 315 may enter and/or exit the tube 320 via the opening 322. The tip portion 321 may comprise a conical tip and the opening 322 may define a diameter between 0.02 mm and 3 mm. The ferromagnetic matrix 325 may be within the tip portion 321 and arranged in a flow path of the solution 315, for example, when the solution 315 is within the tube 320.

When the solution 315 comprises one or more magnetic beads, the magnet 340 may be used to magnetize the ferromagnetic matrix 325 such that the magnetized ferromagnetic matrix 325 captures the magnetic beads (e.g., on the surface of the material used in the ferromagnetic matrix 325) within the tip portion 321 as the solution 315 exits the tube 320. For example, the magnet 340 may be moved between a location proximate to (e.g., adjacent to) the tip portion 321 and a location distal from (e.g., away from) the tip portion 321. When the relative location of the magnet 340 with respect to the tip portion 321 is adjusted, for example, a magnetic field proximate to the tip portion 321 may be changed such that the ferromagnetic matrix 325 is magnetizable.

For example, when the magnet 340 is proximate to the tip portion 321 as the solution 315 is removed from the tube 320 via the tip portion 321, the magnetic beads may be attracted to and captured by the ferromagnetic matrix 325. The ferromagnetic matrix 325 may comprise a steel wool, nickel wool, a cobalt wool, an iron wool, another ferromagnetic material wool, a wire (e.g., coiled, intercrossed, etc.) within the tip portion 321, and/or the like.

It is important to use a material for the ferromagnetic matrix 325 that can fit within the tip portion 321, have the proper magnetic properties (e.g., to be magnetized by the magnet 340), and resist corrosion (e.g., compatible with biological solutions). The ferromagnetic matrix 325 may comprise a material that avoids binding DNA molecules/proteins and RNA molecules/proteins. The ferromagnetic matrix 325 may comprise a ferro-magnetic alloy. The ferro-magnetic alloy may comprise steel and/or nickel. For example, the ferromagnetic matrix 325 may comprise a ferromagnetic stainless steel such as a 300 series austenitic stainless steel (e.g., 303 stainless steel, 304/304L stainless steel, 316/316L stainless steel, 321 stainless steel, or 347 stainless steel), a 400 series martensitic stainless steel (e.g., 410 stainless steel, 416 stainless steel, or 440C stainless steel), or a duplex stainless steel (e.g., 2205/2207 stainless steel).The magnetic strength of the ferromagnetic matrix 325 may be configured to enable a longer release of magnetism. The ferromagnetic matrix 325 may be compatible with biological solutions (e.g., DNA). For example, the ferromagnetic matrix 325 may comprise alloys that are resistant to corrosion (e.g., such as ferromagnetic stainless steels as described herein). In examples, the ferromagnetic matrix 325 may comprise a coating that is configured to prevent binding of DNA molecules/proteins and RNA molecules/proteins. The ferromagnetic matrix 325 in the tip portion 321 may enable re-suspension of particles, which is required in several steps of library preparation. For example, the magnet 340 may be moved away (e.g., distal) from the tip portion 321 such that the magnetic beads are re-suspended in the solution 315.

Although the tip portion 321 is shown in FIG. 3A as part of the tube 320, it should be appreciated that the tip portion 321 may be a replaceable portion of the tube 320 such that the tip portion 321 and the ferromagnetic matrix 325 may be removed and replaced with a different tip portion 321.

When the ferromagnetic matrix 325 is a steel wool, the amount of steel wool within the tip portion 321 (e.g., the density of the steel wool) may have an effect on magnetic bead capture efficiency and/or liquid retention. Thus, there may be a tradeoff between capture efficiency and liquid retention based on the amount of steel wool within the tip portion 321. The more steel wool that is present inside the tip portion 321 (e.g., the greater the density of steel wool in the tip portion), the higher the capture efficiency may be and the higher liquid retention may be, for example, leading to worse DNA elution yields. A steel wool mass of 5 mg or less may be associated with a density that results in bead loss that is comparable with control and a volume elution that is maximized. Table 1 shows example steel wool masses and volume recovery/yields. In Table 1, the steel wool was nitrogen air dried, and the magnet was a ring magnet.

TABLE 1
SPRI and PCR compatibility-Volume Recovery and Yield for Varying Mass

| | | | SPRI Clean Up | | | | PCR | |
|---|---|---|---|---|---|---|---|---|
| | | | Yield | % of | | | Yield | % of |
| Mass | Concentration | Volume | (ng) | Control | Concentration | Volume | (ng) | Control |
| 30 mg | 0.375 | 16 | 6 | 61.2% | 13.1 | 20 | 262 | 80% |
| 20 mg | 0.429 | 14.6 | 6.2634 | 63.9% | 13.7 | 20 | 274 | 84% |
| 10 mg | 0.404 | 20.5 | 8.282 | 84.5% | 15 | 20 | 300 | 92% |

PCR and tapestation results show no ethanol or contaminants carry over when a ferromagnetic matrix 325 is within the tip portion 321. Enriched libraries may be generated and/or sequenced on sequencing systems (e.g., such as the NextSeq 550) using the ferromagnetic matrix 325 within the tip portion 321. Increasing steel wool mass of the ferromagnetic matrix 325 may adversely affect secondary metrics. For example, padded reach enrichment may be decreased when steel wool mass is increased, mean target coverage depth may be decreased when steel wool mass is increased, and uniformity of coverage may be decreased as steel wool mass is increased.

The use of steel wool within the tip portion 321 has the potential to introduce metal contamination from the steel wool, potential lower volumes recover due to surface tension, potential fluidic impedance and manufacturability of tips. However, analyses performed over different samples post enrichment stringent washes (heated washes, NaOH) showed no trace of metal contamination from the use of steel wool in the tip portion 321 to capture magnetic beads. In addition, elution volumes can be fully recovered by blowing compressed air or Nitrogen inside the tip. Blowing compressed air inside the opening 322 of the tip portion 321 may also decrease the Ethanol drying time which reduces the chance for ethanol carryover contamination into polymerase chain reaction (PCR). Additionally or alternatively, a hydrophobic or super-hydrophobic coating on the steel wool may be used to improve volume elution.

Steel wool having a mass of 5-10 mg may result in a negligible fluid impedance when compared to a control. The ferromagnetic matrix 325 may comprise a single wire. The wire may be folded several times, for example, over 2-3 mm lengths in a figure eight shape. The ferromagnetic matrix 325 may comprise nickel wool (e.g., having 99.5% purity). Nickel wool may successfully capture magnetic beads. Steel microbeads with different sizes may be captured using the ferromagnetic matrix 325. Steel wool may reduce the magnetic strength needed for efficient capture, and therefore may reduce an electrical current needed to induce the described magnetic field and/or reduce a size of the magnet 340 (e.g., permanent magnet). Although not shown in the figures, it should be appreciated that other magnetizable materials and/or configurations which provide increased surface area, namely porous sintered metals, 3D printed metal structures, arrays of metal pillars, etc. may be used in

A ferromagnetic matrix magnetic bead system may include an electromagnetic coil. For example, a ferromagnetic matrix, such as stainless-steel wool, incorporated inside a pipette tip may be magnetized by the electromagnetic coil. Control of electric current through the electromagnetic coil may be used to adjust the magnetic field created by the electromagnetic coil. That is, the electromagnetic coil may remain stationary as the magnetic field is adjusted to capture and re-suspend magnetic beads. Similar to the embodiment of FIG. 3A, electromagnetic coil ferromagnetic matrix magnetization may be temporary and may be reverted when the flow of electric current is interrupted. The paramagnetic beads may be resuspended (e.g., fully resuspended) within a solution when the external magnet is removed. The reversibility of the magnetization and bead capture may be desirable for library preparation workflows where beads need to be captured and resuspended. Re-use of magnetic beads may decrease library preparation time and reduce laboratory storage requirements.

FIG. 3B illustrates a cross-section view of another example flow-through magnetic bead processing system 300B. The flow-through magnetic bead processing system 300B may comprise a reservoir 310, a tube 320, a ferromagnetic matrix 325, and an electromagnetic coil 350. The reservoir 310 may be configured to receive and/or retain a solution 315 used in one or more DNA sequencing procedures, for example, from the tube 320. One or more magnetic beads (e.g., such as magnetic bead 130 shown in FIG. 1) may be suspended in the solution 315 to perform the one or more DNA sequencing procedures. For example, the magnetic beads may be used in DNA purification, DNA size selection, RNA Isolation, RNA Purification, Tagmentation, Enrichment, and/or Normalization. The tube 320 may define a tip portion 321. The tube 320 may define an opening 322 in the tip portion 321. The opening 322 may be configured to receive the solution 315. For example, the solution 315 may enter and/or exit the tube 320 via the opening 322. The ferromagnetic matrix 325 may be within the tip portion 321 and arranged in a flow path of the solution 315, for example, when the solution 315 is within the tube 320. It is important to use a material for the ferromagnetic matrix 325 that can fit within the tip portion, have the proper magnetic properties, and resist corrosion (e.g., compatible with biological solutions). For example, the ferromagnetic matrix 325 may comprise a steel wool or a nickel wool.

When the solution 315 comprises one or more magnetic beads, the electromagnetic coil 350 may be used to magnetize the ferromagnetic matrix 325 such that the magnetized ferromagnetic matrix 325 captures the magnetic beads within the tip portion 321 as the solution 315 exits the tube 320. For example, the electromagnetic coil 350 may be wound around the tip portion 321. A flow of current may be introduced to the electromagnetic coil 350, for example, via one or more electrical leads 355. When current is flowing through the electromagnetic coil 350, a magnetic field may be applied proximate to the tip portion 321 such that the ferromagnetic matrix 325 is magnetizable.

For example, when current is flowing through the electromagnetic coil 350 as the solution 315 is removed from the tube 320 via the tip portion 321, the magnetic beads may be attracted to and captured by the ferromagnetic matrix 325. The magnetic field proximate to the tip portion 321 may be removed by interrupting a flow of current through the electromagnetic coil 350. The ferromagnetic matrix 325 may comprise a steel wool, nickel wool, a cobalt wool, an iron wool, another ferromagnetic material wool, a wire (e.g., coiled, intercrossed, etc.) within the tip portion 321, and/or the like.

Control of electric current through the electromagnetic coil 350 may be used to adjust the magnetic field created by the electromagnetic coil 350. That is, the electromagnetic coil 350 may remain stationary as the magnetic field is adjusted to capture and re-suspend magnetic beads. Similar to the embodiment of FIG. 3A, electromagnetic coil 350 ferromagnetic matrix 325 magnetization may be temporary and may be reverted when the flow of electric current is interrupted. For example, the ferromagnetic matrix 325 may lose magnetization when the electric current flowing through the electromagnetic coil 350 is interrupted. The paramagnetic beads may be resuspended (e.g., fully resuspended) within a solution when the flow of electric current through the electromagnetic coil 350 is interrupted. The reversibility of the magnetization and bead capture may be desirable for library preparation workflows where beads need to be captured and resuspended. Re-use of magnetic beads may decrease library preparation time and reduce laboratory storage requirements.

A tube with a magnetizable layer may be used for paramagnetic bead capture. For example, a ferromagnetic layer, such as stainless-steel, may be incorporated on an inside surface of a pipette tip. Additionally or alternatively, the entire tip portion and/or tube may be made of the ferromagnetic material. The magnetizable layer may be magnetized by an external magnet and/or an electromagnetic coil. When the magnetizable layer is magnetized, paramagnetic beads may be captured on the inner surface of the tip portion. Magnetization of the magnetizable layer may be temporary and may be reverted when the external magnet is removed (e.g., located distal from the pipette tip) and/or when a flow of current through the electromagnetic coil is interrupted. The paramagnetic beads may be resuspended (e.g., fully resuspended) within a solution when the external magnet is removed and/or when the flow of current through the electromagnetic coil is interrupted. The reversibility of the magnetization and bead capture may be desirable for library preparation workflows where beads need to be captured and resuspended. Re-use of magnetic beads may decrease library preparation time and reduce laboratory storage requirements.

FIG. 3C illustrates a cross-section view of an example flow-through magnetic bead processing system 300C. The flow-through magnetic bead processing system 300C may comprise a reservoir 310, a tube 320, and a magnet 340. The reservoir 310 may be configured to receive and/or retain a solution 315 used in one or more DNA sequencing procedures, for example, from the tube 320. One or more magnetic beads (e.g., such as magnetic bead 130 shown in FIG. 1) may be suspended in the solution 315 to perform the one or more DNA sequencing procedures. For example, the magnetic beads may be used in DNA purification, DNA size selection, RNA Isolation, RNA Purification, Tagmentation, Enrichment, and/or Normalization. The tube 320 may define a tip portion 321. The tube 320 may define an opening 322 in the tip portion 321. The opening 322 may be configured to receive the solution 315. For example, the solution 315 may enter and/or exit the tube 320 via the opening 322. The tip portion 321 may comprise a magnetizable material layer 335 applied to an inner surface 323 of the tip portion 321. The magnetizable material layer 335 may be within the tip portion 321 and arranged in a flow path of the solution 315, for example, when the solution 315 is within the tube 320. The magnetizable material layer 335 may comprise a ferromagnetic stainless steel. The ferromagnetic stainless steel may comprise a 416 grade stainless steel.

When the solution 315 comprises one or more magnetic beads, the magnet 340 may be used to magnetize the magnetizable material layer 335 such that the magnetic beads are captured within the tip portion 321 (e.g., against the magnetizable material layer 335 as the solution 315 exits the tube 320. For example, the magnet 340 may be moved between a location proximate to (e.g., adjacent to) the tip portion 321 and a location distal from (e.g., away from) the tip portion 321. When the relative location of the magnet 340 with respect to the tip portion 321 is adjusted, for example, a magnetic field proximate to the tip portion 321 may be changed such that the magnetizable material layer 335 is magnetized and de-magnetized.

For example, when the magnet 340 is proximate to the tip portion 321 as the solution 315 is removed from the tube 320 via the tip portion 321, the magnetic beads may be attracted to and captured by the magnetizable material layer 335. The magnetizable material layer 335 may comprise a steel wool, a nickel wool, a cobalt wool, an iron wool, another ferromagnetic material wool, a wire (e.g., coiled, intercrossed, etc.) within the tip portion 321, and/or the like. Although FIG. 3C depicts the inner surface 323 of the tip portion 321 as comprising the magnetizable material layer 335, it should be appreciated that the entire tube 320 may be made from a magnetizable material in the flow-through magnetic bead processing system 300C.

Magnetizing the magnetizable material layer 335 in the tip portion 321 may result in faster magnetic bead capture than using a ferromagnetic matrix (e.g., such as the ferromagnetic matrix 325 shown in FIGS. 3A, 3B). Because the magnetizable material layer 335 is in close contact with the magnetic beads, the magnetized surface area may be high enough for optimal capture of magnetic beads. A tube 320 with a magnetizable material layer 335 may be capable of accommodating larger magnets between tube/needle racks compares to standard tube tips. Steel tubes/needles may be washed and reused several times to reduce plastic waste. Clogs may be a concern in a tube 320 with a magnetizable material layer 335. Different tube sizes may be used for different applications, for example, to avoid magnetic bead clogging. Although FIG. 3C shows the tip portion 321 as having a cylindrical shape, it should be appreciated that any shaped tip portion 321 that comprises a magnetizable material layer 335 may be used in the flow-through magnetic bead processing system 300C, for instance, the tip portion 321 may be conical or pipette tip shaped.

A magnet-in-tip system may be used for paramagnetic bead capture. For example, a magnet, such as a permanent magnet, may be incorporated inside a pipette tip. The magnet may be within the flow path of a magnetic bead suspension. Paramagnetic beads may be captured on the surface of the magnet, for example, as the solution is removed from the pipette tip.

FIG. 3D illustrates a cross-section view of an example flow-through magnetic bead processing system 300D. The flow-through magnetic bead processing system 300D may comprise a reservoir 310, a tube 330, and a magnet 345. The magnet 345 may be a permanent magnet, such as a rare earth magnet. The magnet 345 may be spherical, as shown. The reservoir 310 may be configured to receive and/or retain a solution 315 used in one or more DNA sequencing procedures, for example, from the tube 330. One or more magnetic beads (e.g., such as magnetic bead 130 shown in FIG. 1) may be suspended in the solution 315 to perform the one or more DNA sequencing procedures. For example, the magnetic beads may be used in DNA purification, DNA size selection, RNA Isolation, RNA Purification, Tagmentation, Enrichment, and/or Normalization.

The tube 330 may define a diameter D1. The tube 330 may define a tip portion 331. The tube 330 may define an opening 332 in the tip portion 331. The opening 332 may be configured to receive the solution 315. For example, the solution 315 may enter and/or exit the tube 330 via the opening 332. The opening 332 may define a diameter D2. The tip portion 331 may be conical, for example, such that the tip portion 331 tapers from the diameter D1 to the diameter D2. The magnet 345 may be received within the tip portion 331. For example, the magnet 345 may be arranged in a flow path of the solution 315, for example, when the solution 315 is within the tube 330. The magnet 345 may define a diameter D3. The diameter D3 of the magnet may be greater than the diameter D2 of the opening 332 and less than the diameter D1 of the tube 330. The conical shape of the tip portion 331 may keep the magnet within the tube 330 and/or prevent the magnet 345 from exiting the tube 330 via the opening 332.

When the solution 315 comprises one or more magnetic beads, the magnet 345 may attract and capture the magnetic beads within the tip portion 331 as the solution 315 exits the tube 330. For example, the magnetic beads may be captured around the surface of the magnet 345. The tube 330 may be moved around to optimize bead capture. For example, moving the tube 330 around may enable the magnet 345 to come in contact with a greater percentage of the solution 315. Clogs may be a concern in a tube 330 with a magnet 345 in the flow path. For example, magnetic beads may accumulate on the magnet 345 such that the flow path through the tip portion 331 is blocked. Different tube sizes may be used for different applications, for example, to avoid magnetic bead clogging. Although FIG. 3D shows the magnet 345 as having a spherical shape, it should be appreciated that the magnet 345 may have an alternate shape.

The magnet 345 in the tip portion 331 may be used for SPRI clean-up which may result in comparable performances compared to manual standard. The magnet 345 in the tip portion 331 may avoid metal contamination after DNA elution.

Current requirements of one or more library prep steps may be incompatible with the magnet 345 in the tip portion 331, for example, due to the need of beads resuspension during stringency washes (58C, buffer). Enrichment experiments show that magnet-in-tip did not provide adequate bead washing. PCR failed after enrichment, likely due to carryover of a PCR inhibitor and/or incomplete elution of DNA. A magnet in-tip system (e.g., such as the magnetic bead processing system 300D shown in FIG. 3D) may remain a valid candidate for workflows where bead resuspension is not required.

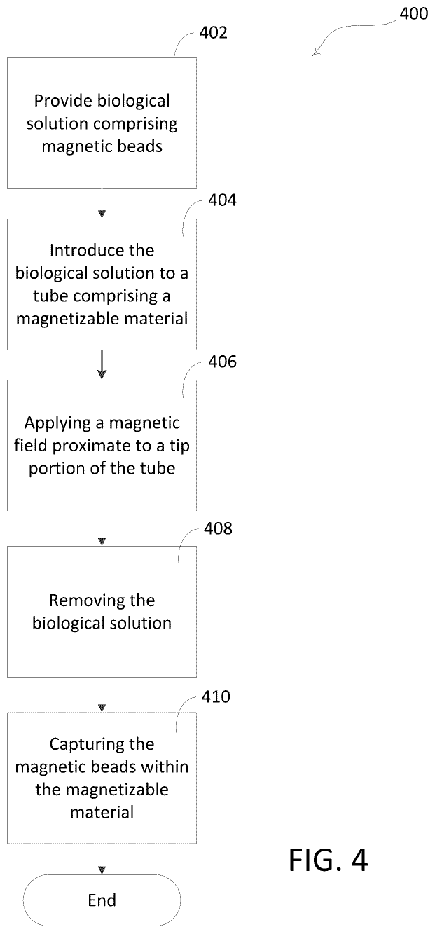

FIG. 4 depicts an example process 400 of using an in-tip flow-through magnetic bead processing system (e.g., such as the flow-through magnetic bead processing system 300A shown in FIG. 3A, the flow-through magnetic bead processing system 300B shown in FIG. 3B, the flow-through magnetic bead processing system 300C shown in FIG. 3C, and/or the flow-through magnetic bead processing system 300D shown in FIG. 3D). The in-tip flow-through magnetic bead processing system may comprise a magnet-in-tip configuration, a magnetizable material in the solution configuration, a ferromagnetic matrix in tip configuration, and/or a ferromagnetic tube surface configuration. The ferromagnetic matrix magnetic bead system may comprise a tube having a tube tip, a ferromagnetic matrix within the tube tip, and an external magnet. The ferromagnetic matrix magnetic bead system may comprise a computing device (e.g., such as the computing device 1000 shown in FIG. 5). The example process 400 may be used for one or more DNA sequencing procedures, for example, such as DNA purification, DNA size selection, RNA Isolation, RNA Purification, Tagmentation, Enrichment, and/or Normalization. The example process 400 may be manual, automated, or a mix of manual steps and automated steps.

At 402, a biological solution comprising a plurality of magnetic beads suspended therein may be provided. The biological solution may be associated with one or more DNA sequencing procedures. The biological solution may be a solution that comprises DNA. The biological solution may comprise a plurality of magnetic beads.

At 404, the biological solution may be introduced to the tube of the ferromagnetic matrix magnetic bead system. The tube may comprise an opening in a tip portion of the tube. The biological solution may be received into the tube via the opening. The tip portion of the tube may comprise a magnetizable material arranged in a flow path of the biological solution. The magnetizable material may comprise a ferromagnetic matrix, a wire (e.g., coiled, intercrossed, etc.) within the tip portion, or a magnetizable material layer applied to an inner surface of the tip portion. It is important to use a material for the magnetizable material that can fit within the tip portion, have the proper magnetic properties, and resist corrosion (e.g., compatible with biological solutions). The ferromagnetic matrix may comprise a steel wool or a nickel wool (e.g., with 99.5% purity). The ferromagnetic matrix (e.g., steel or nickel wool) may comprise a mass of 10 mg or less. Steel wool masses below 20 mg may increase yield and volume recovery. The ferromagnetic matrix may comprise a nickel wool. When the magnetizable material comprises a wire (e.g., coiled, intercrossed, etc.) within the tip portion, the wire may be folded over a plurality of times across the tip portion (e.g., such as in a figure eight shape).

At 406, a magnetic field may be applied proximate to the tip portion of the tube. The magnetic field may be applied using an electromagnetic coil and/or an external magnet. When the magnetic field is applied using an electromagnetic coil, the electromagnetic coil may be wound around an external surface of the tip portion and a current may be introduced to the electromagnetic coil. When the magnetic field is applied using an external magnet, the external magnet may be moved closer to the tip portion (e.g., an external surface of the tip portion) of the tube. For example, the external magnet may be moved from a location distal from the tip portion to a location proximate to the tip portion. When the magnetic field is applied at 406, the magnetizable material may become magnetized and may attract magnetic beads that are proximate to the magnetizable material within the solution.

At 408, the biological solution may be removed from the tube, for example, via the opening in the tip portion (e.g., to enable magnetic re-suspension, magnetic bead washing, and/or another process). As the biological solution exits the tube, the biological solution may flow across the magnetizable material. The magnetizable material may be magnetized as the biological solution exits the tube and flows across the magnetizable material.

At 410, the magnetizable material may capture the magnetic beads, for example, as the biological solution exits the tube. For example, magnetic beads that initially were not proximate to the magnetizable material may be captured by (e.g., within) the magnetizable material as the biological solution is removed from the tube. The plurality of magnetic beads suspended in the biological solution may be captured in the magnetizable material in the tip portion using the magnetic field. For example, the magnetic field may magnetize the magnetizable material and the magnetic beads may be attracted to the magnetizable material such that the magnetic beads are captured by the magnetizable material as the biological solution is removed from the tube.

While the magnetic beads are captured, at 410, by the magnetizable material. The magnetic field proximate to the tip portion may be decreased and/or removed. For example, the magnetic field proximate to the tip portion may be decreased as an external magnet is moved away from the tip portion and/or as a flow of current through a magnetic coil is decreased. The magnetic field proximate to the tip portion may be removed (e.g., to re-suspend the magnetic beads) when the external magnet is moved away far enough that its magnetic field does not contact the tip portion and/or by interrupting a flow of current through the electromagnetic coil.

A second solution (e.g., such as a buffer solution) may be introduced into the tube via the opening in the tip portion such that the captured plurality of magnetic beads are suspended in the second solution. For example, the second solution may be introduced to the tube when the magnetic field is removed from the tip portion of the tube. The second solution may flow across the magnetizable material, for example, when entering the tip portion. As the second solution flows across the magnetizable material, the second solution may abut the magnetic beads captured in the magnetizable material such that they become suspended in the second solution.

The magnetic beads may be solid phase reversible immobilization (SPRI) magnetic beads, streptavidin magnetic beads (SMBs), AMPure XP beads, sample purification beads, Illumina purification beads, RNA purification beads, rRNA removal beads, RNAClean XP beads, Bead-linked Transposomes, library normalization beads, equalizer beads, and/or another type of magnetic beads. Although the different types of magnetic beads often look similar, each type of magnetic bead may have different properties and functions. When used in library preparation, the magnetic beads may be brought to room temperature before use (e.g., for approximately 30 minutes). The magnetic beads may be pulse-vortexed for 1 full minute, for example, to ensure homogenous resuspension. The magnetic beads may be stored at an appropriate temperature when not in use. Each type of magnetic beads may have specific storage requirements.

Purification beads (e.g., AMPure XP beads, sample purification beads, Illumina purification beads, etc.) may be used for DNA purification and/or size selection of DNA fragments based on the ratio of beads. RNA Isolation and Purification beads (e.g., RNA purification beads, rRNA removal beads, etc.) may be used to isolate mRNA by capturing polyadenylated RNAs and/or remove rRNA by binding rRNA removal probes bound to rRNA. Tagmentation beads (e.g., bead-linked transposomes, etc.) may comprise transposomes bound to beads to fragment and tag (e.g., tagment) DNA input, for example, for subsequent enrichment, for DNA PCR-free workflow, and/or the like. Enrichment beads (e.g., streptavidin magnetic beads, etc.) may comprise streptavidin bound to magnetic beads to pull down biotinylated capture probes. Normalization beads (e.g., library normalization beads, etc.) may comprise a fixed volume of beads added to each sample. The normalization beads may bind and saturate with library fragments such that excess library is washed away; and, a normalized library may be eluted from the normalization beads. Additionally or alternatively, normalization beads (e.g., equalizer beads, etc.) may be added to a sample before or after an equalizer capture reagent is added to final library(ies). When library fragments are captured by the normalization beads, the normalization beads may be washed and a normalized library may be eluted from the normalization beads. The magnetic beads may be of various sizes. For example, the magnetic beads may define a diameter in the range of 100 nanometers to 10 micrometers. A single size of magnetic beads may be selected for a specific process or group of processes. The magnetic beads may be sized to fit into the wells of the microtiter and/or picotiter plates.

Magnetizable material may be added to reagents, for example, for faster paramagnetic bead capture. In this approach, micron size magnetite particles may be mixed in suspension with small paramagnetic beads. When an external magnetic field is applied (e.g., via permanent magnet or electromagnetic coil), the magnetite particles may migrate faster to form a pellet. The larger the paramagnetic beads, the faster the pull down time. The interaction between big and small beads favor bead separation. Bovine Serum Albumin (BSA) blocking the magnetite particles may enable near parity of DNA elution with standard SPRI. A coating may be applied to the magnetite particles, for example to avoid contamination. Core-shell particles with a magnetic core and Silica shell may be used as the magnetizable material. A magnetic complex may be provided where larger magnetic particles are pre-complexed with smaller magnetic particles (e.g., via magnetic forces), allowing high surface area for capture as well as improved ease of pulldown.

FIG. 5 illustrates a block diagram of an example computing device 1000. One or more computing devices such as the computing device 1000 may implement one or more features of the flow-through magnetic bead processing systems described herein. For example, the computing device 1000 may be configured to control movement of an external magnet (e.g., such as the magnet 340 shown in FIGS. 3A and 3C), introduction and evacuation of one or more solutions from a tube (e.g., such as the tube 320 shown in FIGS. 3A-3C and/or the tube 330 shown in FIG. 3D), a flow of current through an electromagnetic coil (e.g., such as the electromagnetic coil 350 shown in FIG. 3B), and/or the like. Additionally or alternatively, one or more computing devices, such as the computing device 1000 may be used to implement at least a portion of the process 400 shown in FIG. 4. As shown by FIG. 5, the computing device 1000 may comprise a processor 1002, a memory 1004, a storage device 1006, an I/O interface 1008, and a communication interface 1010, which may be communicatively coupled by way of a communication infrastructure 1012. It should be appreciated that the computing device 1000 may include fewer or more components than those shown in FIG. 5.

The processor 1002 may include hardware for executing instructions, such as those making up a computer program. In examples, to execute instructions for dynamically modifying workflows, the processor 1002 may retrieve (or fetch) the instructions from an internal register, an internal cache, the memory 1004, or the storage device 1006 and decode and execute the instructions. The memory 1004 may be a volatile or non-volatile memory used for storing data, metadata, and programs for execution by the processor(s). The storage device 1006 may include storage, such as a hard disk, flash disk drive, or other digital storage device, for storing data or instructions for performing the methods described herein.

The I/O interface 1008 may allow a user to provide input to, receive output from, and/or otherwise transfer data to and receive data from the computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. The I/O interface 1008 may be configured to provide graphical data to a display for presentation to a user. The graphical data may be representative of one or more graphical user interfaces and/or any other graphical content.

The communication interface 1010 may include hardware, software, or both. In any event, the communication interface 1010 may provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the library prep and/or sequencing processes may allow a plurality of devices (e.g., a client device, sequencing device, and server device(s)) to exchange information such as library prep data, sequencing data, and/or error notifications.

What is claimed is:

1. A method comprising:
providing a biological solution comprising a plurality of magnetic beads suspended therein;
introducing the biological solution to a tube via an opening in a tip portion of the tube, the tip portion of the tube comprising a magnetizable material arranged in a flow path of the biological solution;
applying a magnetic field proximate to the tip portion of the tube using an electromagnetic coil;
removing the biological solution from the tube via the opening in the tip portion; and
capturing the plurality of magnetic beads within the magnetizable material in the tip portion using the magnetic field.

2. The method of claim 1, wherein the magnetizable material comprises a ferromagnetic matrix.

3. The method of claim 2, wherein the ferromagnetic matrix comprises a steel wool.

4. The method of claim 3, wherein the steel wool comprises a mass of 20 mg or less.

5. The method of claim 2, wherein the ferromagnetic matrix comprises a nickel wool.

6. The method of claim 1, wherein the magnetizable material comprises a wire that is coiled within the tip portion.

7. The method of claim 1, wherein the electromagnetic coil is wound around the tip portion.

8. The method of claim 1, wherein the magnetic field is applied proximate to the tip portion by introducing a flow of current through the electromagnetic coil.

9. The method of claim 1, further comprising removing the magnetic field proximate to the tip portion by interrupting a flow of current through the electromagnetic coil.

10. The method of claim 9, further comprising introducing a second solution to the tube via the opening in the tip portion such that the captured plurality of magnetic beads are suspended in the second solution.

11-34. (canceled)

35. A flow-through magnetic bead processing system, comprising:
a reservoir configured to receive a biological solution comprising a plurality of magnetic beads suspended therein;
a tube defining a tip portion having an opening, the opening configured to introduce the biological solution from the reservoir to the tube;
a magnetizable material within the tip portion, the magnetizable material arranged in a flow path of the biological solution; and
an electromagnetic coil configured to apply a magnetic field proximate to the tip portion of the tube,
wherein the magnetizable material is configured to capture the plurality of magnetic beads when the biological solution is removed from the tube via the opening in the tip portion.

36. The flow-through magnetic bead processing system of claim 35, wherein the magnetizable material comprises a ferromagnetic matrix.

37. The flow-through magnetic bead processing system of claim 36, wherein the ferromagnetic matrix comprises a steel wool.

38. The flow-through magnetic bead processing system of claim 37, wherein the steel wool comprises a mass of 20 mg or less.

39. The flow-through magnetic bead processing system of claim 36, wherein the ferromagnetic matrix comprises a nickel wool.

40. The flow-through magnetic bead processing system of claim 35, wherein the magnetizable material comprises a wire that is coiled within the tip portion.

41. The flow-through magnetic bead processing system of claim 35, wherein the electromagnetic coil is wound around the tip portion.

42. The flow-through magnetic bead processing system of claim 35, wherein the magnetic field is applied proximate to the tip portion by introducing a flow of current through the electromagnetic coil.

43. The flow-through magnetic bead processing system of claim 35, wherein the magnetic field is removed from tip portion by interrupting a flow of current through the electromagnetic coil.

44. The flow-through magnetic bead processing system of claim 43, wherein a second solution is introduced to the tube via the opening in the tip portion after the biological solution is removed from the tube such that the captured plurality of magnetic beads are suspended in the second solution.
